(19) United States
(12) Patent Application Publication
(10) Pub. No.: US 2012/0082709 A1
(43) Pub. Date: Apr. 5, 2012

(54) COMPOSITIONS AND METHODS FOR DELIVERY OF MATERIALS

(75) Inventors: David D. Taft, Menlo Park, CA (US); Stephen P. Bitler, Menlo Park, CA (US); Qiang Zheng, Palo Alto, CA (US); Natarajan Balanchander, Menlo Park, CA (US); Sandra N. Ottensman, Menlo Park, CA (US)

(73) Assignee: Landec Corporation, Meno Park, CA (US)

(21) Appl. No.: 13/377,566

(22) PCT Filed: Jun. 11, 2010
(86) PCT No.: PCT/US2010/038239

§ 371 Date: Dec. 12, 2011

Related U.S. Application Data
(60) Provisional application No. 61/268,329, filed on Jun. 11, 2009.

(51) Int. Cl. A61K 47/36; A61K 8/02; A01N 25/10; A61K 8/65; A61K 47/42; A61K 47/40; A61K 8/73; A61K 47/08; A61K 9/14

(52) U.S. Cl. 424/401; 514/772; 424/405; 424/489; 514/774; 514/773; 514/777

(57) ABSTRACT

Compositions for delivering release materials, for example drugs, agricultural materials, other bioactive materials, cosmetic materials, organic or inorganic compounds which take part in or catalyse a chemical reaction, a fragrance-emitting material for a vaporizer, or a lubricating material. The release material is associated with a crystalline carrier. The carrier may be a crystalline polymer containing a plurality of crystalline groups or a crystalline monomer which contains a single crystalline group, the crystalline group preferably comprising a straight chain polymethylene radical containing at least 16 carbon atoms. Some of the carriers are obtained by modifying hyaluronic acid, collagen, gelatin, a polysaccharide, a carbohydrate or cyclodextran. The release material may contain a similar crystalline group, for example may be an active compound which has been modified to introduce a crystalline group.

### CROSS-REFERENCE TO RELATED APPLICATIONS

This application claims the benefit of and priority to U.S. provisional application 61/268,329, filed Jun. 11, 2009 (Docket 009 PRV).

This application is related to

(1) U.S. provisional Application No. 60/873,234, filed Dec. 5, 2006 (Docket 001 PRV),

(2) U.S. application Ser. No. 11/999,415, filed Dec. 4, 2007 (Docket 001 US), which claims priority from (1), published as US 2008-026 11 9105

(3) U.S. provisional Application Nos. 61/005,400, filed Dec. 4, 2007 (Docket 003 PRV),

(4) International Patent Application PCT/US 2007/025032 (Docket 002 PCT), which was (i) filed Dec. 4, 2007, claiming priority from (1), and (ii) published as WO/2008/070165 on Jun. 12, 2008,

(5) International Patent Application PCT/US 2007/024909 (Docket 002 PCT), which was (i) filed Dec. 4, 2007, claiming priority from (1), and (ii) published as WO/2008/0701 18 on Jun. 12, 2008,

(6) U.S. provisional application 61/131,123, filed Jun. 4, 2008 (Docket 004 PRV),

(7) U.S. provisional application 61/131,716, filed Jun. 10, 2008 (Docket 004B PRV),

(8) U.S. application Ser. No. 12/284,755, filed Sep. 25, 2008 (Docket 005 US), which is a continuation of U.S. application Ser. No. 11/999,415, and which claims priority from (3) and (6),

(9) U.S. application Ser. No. 12/287,520 filed Oct. 10, 2008 (Docket 006 US), which claims priority from (3), (6) and (7), published as US 2009-0209558

(10) International Patent Application PCT/US 2008/013335 (Docket 007 PCT), filed Dec. 3, 2008, claiming priority from (3), (6) and (7), and published as WO 2009073192,

(11) U.S. application Ser. No. 12/315,876, filed Dec. 4, 2008 (Docket 008 US), which claims priority from (3), (6) and (7),

(12) U.S. application Ser. No. 12/455,650 filed Jun. 4, 2009, (Docket 011 US), and entitled Compositions and Methods for Agriculture and Aquaculture, and which claims priority from (6) and (7), and which was published as 20100004124,

(13) U.S. application Ser. No. 12/455,628, filed Jun. 4, 2009, (Docket 010 US), and entitled Compositions and Methods for Personal Care, which claims priority from (6) and (7), and which was published as US 200900246155,

(14) the PCT application filed Jun. 4, 2010 (Docket 012 PCT), claiming priority from U.S. Provisional Application No. 61/217,772 (Docket 012 PRV),

(15) the PCT application filed Jun. 4, 2010, (Docket 011 PCT) claiming priority from U.S. application Ser. No. 12/455,650, filed Jun. 4, 2010 (Docket 011 US above).

The entire disclosure of each of the applications and publications identified above is incorporated by reference herein for all purposes.

The patent applications noted above disclose the use of certain crystalline materials (referred to in the applications identified in (10)-(15) above as “CYC carriers”) for the delivery of useful materials (referred to in the applications identified in (10)-(15) above as “release materials”). The release materials include, but are not limited to, cosmetic materials, agricultural materials, and drugs which are useful in the treatment of human beings or other mammals.

### FIELD OF THE INVENTION

This invention relates to systems for the delivery of useful materials.

### BACKGROUND

There are many known systems for the controlled delivery of useful materials.

### SUMMARY OF THE INVENTION

This specification discloses as an invention further crystalline materials which can be used for the delivery of useful materials, and further useful materials which can be delivered by the CYC carriers disclosed in the earlier cases and/or by the further crystalline materials disclosed herein.

Some of the further crystalline materials disclosed herein fall within the definition of CYC carriers in the earlier cases, but others fall outside that definition. The further crystalline materials that fall within the earlier definition are generally referred to in this specification as “supplementary CYC compounds”, and the further crystalline materials that fall outside the earlier definition are generally referred to in this specification as “mono-Cy CYC compounds”. The mono-Cy CYC compounds are defined below. The term “QCYC carrier” is used in this specification to denote both the CYC carriers as defined in the earlier application and the mono-Cy CYC carriers, unless the context requires otherwise.

Some of the additional classes of useful materials are drugs, cosmetic materials or agricultural materials as defined in the earlier cases, but others fall outside those definitions.

The invention disclosed by this specification also includes compositions which comprise microparticles comprising a QCYC carrier, and, associated therewith, a useful material, some of which compositions are disclosed in the PCT application filed Jun. 4, 2010 (Docket 012 PCT), claiming priority from U.S. Provisional Application No. 61/217,772 (Docket 012 PRV). The particles can for example have an average particle size of 0.5-250μ, e.g. 0.5-150, 10-150, 20-150, 20-70, or 0.5-25μ. Particle sizes given in this specification are measured using a Horiba LA-910 Laser Scattering Particle Size Distribution Analyzer.

The invention disclosed by this specification also includes modified release materials that can be used with or without a QCYC carrier.

The invention disclosed by this specification also includes uses of the QCYC carriers for purposes other than the delivery of release materials.

The CYC carriers disclosed in the earlier cases comprise (i) certain polymers which comprise repeating units containing crystalline moieties and/or terminal units comprising crystalline moieties (those polymers being referred to as CYC polymers), (ii) certain non-polymeric compounds which contain crystalline moieties (those compounds being referred to as CYC compounds), (iii) certain “self-assemblies” which contain crystalline moieties (those assemblies being referred to as “CYC assemblies”).

Definitions of CYC polymers (and CYSC polymers, ECC polymers, and SSP polymers, which are subclasses of CYC polymers), CYC compounds, CYC assemblies and mono-Cy CYC carriers are given below. Where the disclosure below refers to one of the QCYC carriers, a component of one of the QCYC carriers, a characteristic of one of QCYC carriers, or to a composition or method making use of one of the QCYC carriers, that disclosure is also applicable to other QCYC carriers, unless the context requires otherwise.

The term “CYC polymer” is defined herein as a polymer which:

(A) comprises polymeric molecules having a polymer backbone and comprising at least one moiety which

(i) has the formula -b-Cy, and

(ii) either
(A) forms part of a repeating unit of the polymer backbone, the repeating unit having formula (1) below

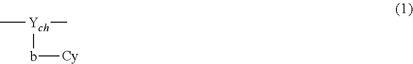

where Y_{ch} is a moiety forming part of the polymer backbone,
b is a bond or moiety which links the Cy moiety to Y_{ch}, and
Cy is a moiety which is associated with other moieties (which may also be Cy moieties) to provide the CYC polymer with crystallinity;
or
(B) forms part of a terminal unit of the polymer backbone, the terminal unit having formula (2) below

-Yₜₑᵣₘ-b-Cy (2)
where Yₜₑᵣₘ is a moiety at the end of the backbone, and
b and Cy are as defined in formula (1); and
   (B) has a crystalline melting temperature (hereinafter abbreviated to Tp) of at least 0° C. and a heat of fusion (hereinafter abbreviated to ΔH) of at least 3 J/g which result from association of the Cy moieties. In this definition, and throughout this specification, Tp and ΔH are measured on a differential scanning calorimeter (DSC) as hereinafter described.

The moiety -b-Cy is also referred to in this specification as an -Rc moiety, i.e. Rc is synonymous with b-Cy.

In some CYC polymers, the backbone of the polymeric molecules comprises repeating units having formula (3) below

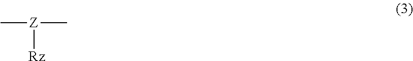

where Z is a moiety forming part of the backbone and Rz represents a moiety which does not comprise a Cy moiety. Many useful CYC polymers of this kind have an amphiphilic character, with the Cy moieties providing hydrophobic characteristics and the Z(Rz) moieties providing hydrophilic characteristics.

The term “CYSC polymer” is used herein to denote a CYC polymer in which at least a majority by weight, preferably at least 90% by weight, particularly substantially all, of the Cy moieties are present in repeating units of formula (1). Thus, a CYSC polymer always contains repeating units of formula (1), and optionally contains terminal units of formula (2) and repeating units of formula (3). Patents and other publications relating to CYSC polymers include J. Poly. Sci. 60, 19 (1962); J. Poly. Sci, (Polymer Chemistry) 7, 3053 (1969), 9, 1835, 3349, 3351, 3367, 10, 1657, 3347, 18, 2197, 19, 1871; J. Poly. Sci, Poly-Physics Ed 18 2197 (1980); J. Poly. Sci, Macromol. Rev, 8, 117 (1974); Macromolecules 12, 94 (1979), 13, 12, 15, 18, 2141, 19, 611; JACS 75, 3326 (1953), 76; 6280; Polymer J 17, 991 (1985); and Poly. Sci USSR 21, 241 (1979); U.S. Pat. Nos. 4,830,855, 5,120,349, 5,129,180, 5,156,911, 5,254,354, 5,387,450, 5,412,035, 5,469,867, 5,665,822, 5,752,926, 5,783,302, 6,013,293, 6,060,540, 6,199,318, 6,210,724, 6,224,793, 6,255,367, 6,376,032, 6,492,462, 6,540,984, 6,548,132, 6,831,116, 6,989,417, and 7,101,928; and US Patent Application Publications Nos. 2001/0018484, 2002/0090425 and 2002/0127305. The entire disclosure of each of those publications, patents and patent publications is incorporated herein by reference for all purposes.

The term “ECC polymer” is used herein to denote a CYC polymer in which at least a majority by weight, preferably at least 90% by weight, particularly substantially all, of the Cy moieties are present in terminal units of formula (2). Thus, an ECC polymer always contains terminal units of formula (2) and repeating units of formula (3), and optionally contains repeating units of formula (1).

The term SSP polymer is used herein to denote a polymer which

(1) has a crystalline melting temperature, Tp, of at least 25° C., e.g. 27-100° C., and a ΔH of at least 5 J/g; and

(2) comprises polymeric molecules having a backbone which comprises
(a) repeating units which do not contain hydrophilic moieties, and have the formula (1) below,

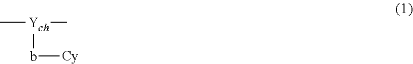

where Y_{ch} is a moiety forming part of the backbone,
   b is a bond or a moiety linking the Cy moiety to Y_{ch}, and
   Cy is a moiety is associated with other Cy moieties to provide the SSP polymer with crystallinity;
(b) repeating units which have the formula (2zphil) below,

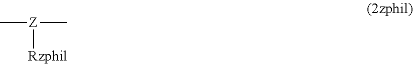

where Z is a moiety forming part of the backbone, and
Rzphil comprises a hydrophilic moiety;

the molar ratio of the units of formula (2zphil) to the units of formula (1) being at least 2.5:1.

The term “CYC compound” is defined herein as a non-polymeric compound which

(A) has the formula

Q(-b-Cy)_{q} (4)
wherein q is least 2, e.g. 3-8,
Q is a moiety having a valence of at least q,
b is a bond or a moiety linking the Cy moiety to the Q moiety, and
Cy is as defined in formula (1), and

(B) has a crystalline melting temperature, Tp, of at least 0° C. and a ΔH of at least 3 J/g which results from association of the Cy moieties.

The term “CYC assembly” is defined herein as an assembly of (i) a polymer which is a CYC polymer as defined above except that the polymer does not necessarily have a Tp of at least 0° C. and a ΔH of at least 4 J/g, and (ii) a compound which contains a Cy moiety and which is intimately mixed with the polymer but is not covalently linked to the polymer, the assembly having a crystalline melting temperature, Tp, of at least 0° C. and a ΔH of at least 3 J/g which results from association of the Cy moieties.

The term “mono-Cy CYC carrier” is defined herein as a non-polymeric compound which:

(A) has the formula.

Q-Cy
wherein Cy is as defined in formula (1) above, and
Q is a moiety which (i) is free of Cy moieties and (ii) contains at least one functional moiety, for example a hydrophilic moiety, thus resulting in a compound which is an amphiphile, or a polar group which can associate with a suitable release material, or a functional group which can form a covalent link with a suitable release material; and

(B) has a crystalline melting temperature, Tp, of least 0° C., e.g. at least 25° C., e.g. 27-100° C., and a ΔH of at least 3 J/g.

For additional disclosure of CYC carriers, reference should be made to the patents and applications incorporated by reference herein.

Supplementary CYC carriers disclosed in this specification includes CYC carriers, in particular ECC-PLGA polymers, which have been modified by covalent bonding to a silicon-containing moiety, for example a hydroxy poly-alkyl siloxane. This modification can for example change the surface tension properties of compositions containing the CYC carriers. Such modification can facilitate the application of the composition, and/or change the release characteristics of the release material. The silicon-containing portion of the CYC carrier can for example facilitate alignment of a film containing the CYC carrier on a substrate, e.g. the skin, and can delay or prevent the release material from migrating towards the substrate. This can be desirable, for example, when contact between the release material and the substrate should be delayed or prevented, for example, when the release material is a harmful antioxidant or other additives.

### DETAILED DESCRIPTION OF THE INVENTION

In this specification:

(1) Reference is made to particular features of the invention (including for example components, ingredients, elements, devices, apparatus, systems, groups, ranges, method steps, test results, etc). It is to be understood that the disclosure of the invention in this specification includes all possible combinations of such particular features. For example, where a particular feature is disclosed in the context of a particular embodiment or claim, that feature can also be used, to the extent appropriate, in the context of other particular embodiments and claims, and in the invention generally.
(2) The singular forms “a”, “an”, and “the” include plural referents unless the context dictates otherwise. Thus, for example, a reference to “a part” includes a plurality of such parts.
(3) The term “comprises” and grammatical equivalents thereof are used to mean that, in addition to the features specifically identified, other features are optionally present. For example a formulation which comprises a QCYC carrier and a drug can contain a single QCYC carrier and a single drug, or two or more QCYC carriers and/or two or more drugs, and optionally contains one or more other ingredients which are not QCYC carriers, for example other ingredients as disclosed herein.
(4) The term “consisting essentially of” and grammatical equivalents thereof are used to mean that, in addition to the features specifically identified, other features may be present which do not materially alter the disclosed and/or claimed invention.
(5) The term “at least” followed by a number is used to denote the start of a range beginning with that number (which may be a range having an upper limit or no upper limit, depending on the variable being defined). For example “at least 1” means 1 or more than 1, and “at least 80%” means 80% or more than 80%.
(6) The term “at most” followed by a number is used to denote the end of a range ending with that number (which may be a range having 1 or 0 as its lower limit, or a range having no lower limit, depending upon the variable being defined). For example, “at most 4” means 4 or less than 4, and “at most 40%” means 40% or less than 40%.
(7) A range written as “(a first number) to (a second number)” or “(a first number)-(a second number)” means a range whose lower limit is the first number and whose upper limit is the second number. For example, “from 8 to 20 carbon atoms” or “8-20 carbon atoms” means a range whose lower limit is 8 carbon atoms, and whose upper limit is 20 carbon atoms.
(8) The terms “plural”, “multiple”, “plurality” and “multiplicity” are used herein to denote two or more than two features.
(9) When a method is described as comprising two or more defined steps, the defined steps can be carried out in any order or simultaneously (except where the context excludes that possibility), and the method can optionally include one or more other steps which are carried out before any of the defined steps, between two of the defined steps, or after all the defined steps (except where the context excludes that possibility).
(10) When reference is made to two or more features, this includes the possibility that the two or more features are replaced by a lesser number or greater number of features providing the same function (except where the context excludes that possibility).
(11) The numbers given should be construed with the latitude appropriate to their context and expression; for example, each number is subject to variation which depends on the accuracy with which it can be measured by methods conventionally used by those skilled in the art.
(12) Parts, ratios and percentages are by weight, except where otherwise noted.
(13) Temperatures are in degrees Centigrade (° C.).
(14) Molecular weights of polymers are in Daltons; are number average molecular weights (Mn) unless stated to be weight average molecular weights (Mw); and are measured by gel permeation chromatography (GPC) with a light scattering detection method, for example using a DAWN DSP laser photometer from Wyatt Technology, unless stated to be measured using GPC against a polystyrene standard.
(15) The terms “melting point” (often abbreviated to Tp), “onset of melting temperature” (often abbreviated to To) and “heat of fusion” (which is a measure of the crystallinity of the polymer, is expressed in J/g and is often abbreviated to ΔH) are well known to polymer technologists and refer to quantities determined using a differential scanning calorimeter (hereinafter DSC), e.g. a Q 100 DSC from TA Instruments, at a rate of temperature change of 10° C./min, e.g. from −10 to 150° C., unless otherwise noted. Tp is the peak melting temperature, To is the temperature at the intersection of the baseline of the DSC peak and the onset line, the onset line being defined as the tangent to the steepest part of the DSC curve below Tp, and ΔH is the heat of fusion associated with the endotherm or exotherm as calculated by the DSC and is reported in J/g. Unless otherwise noted, the values of Tp, To and ΔH are measured on the second heat cycle.
(16) Bulk viscosities are given in centipoise and are measured using a Brookfield LVT viscometer with an electronically thermostat controlled thermal heater, controlled for example to about 95° C., and small sample adapter using spindles 4 and 7, or, in the Examples below, spindle 18.
(17) Solubility parameters are calculated using the method described in D. W. van Krevelen, “Properties of Polymers” Elsevier, 1997, p. 200-214 especially p. 214 and reported in J^{1/2}/cm^{3/2}.
(18) The term “associated” and grammatical variations thereof include any type of interaction, including chemical bonds (for example, covalent, ionic and hydrogen bonds) and/or Van der Waals forces, and/or polar and non-polar interactions through other physical constraints provided by molecular structure, and interactions through physical mixing.
(19) The term “pharmaceutical formulation” means a composition which (i) is suitable for administration to a human being or other mammal or which can be treated, e.g. sterilized, to make it suitable for such administration, and (ii) comprises at least one drug and at least one QCYC carrier. The term “drug” means a material which is biologically active in a human being or other mammal, locally and/or systemically.
(20) The term “therapeutically effective amount” or “therapeutically effective dosage” means an amount of a drug which results in a desired therapeutic effect.
(21) The term “agricultural” refers to compositions and methods which are useful in the treatment of plants, seeds and soil. The term “aquacultural” refers to compositions and methods which are useful in the treatment of fresh water or salt water organisms.
(22) The term “Personal Care” refers to compositions and methods which are useful in the treatment of the human body (including human hair) primarily or exclusively for non-medical purposes, for example for cosmetic or hygienic purposes.
(23) The term “organism” includes, but is not limited to, human beings and other mammals, living tissue which is not part of a mammal, freshwater organisms, saltwater organisms, plants, seeds, and soil which contains living organisms. The invention is useful, for example, for delivering drugs to human beings and other mammals; for delivering biocides and/or fertilizers to plants, seeds and soil; for delivering cosmetic ingredients to human beings; and for use as components in cosmetic and other formulations to provide benefits such as thickening and control of rheological properties.
(24) The term “therapeutically effective amount” means an amount of a drug which produces a desired therapeutic effect.
(25) The term “diagnostic agent” means any chemical moiety that can be used for diagnosis or in a diagnostic test. For example, diagnostic agents include imaging agents containing radioisotopes, contrasting agents containing for example iodine, enzymes, fluorescent substances and the like.
(26) The term “treatment” means administration of a composition to a site, e.g. the administration of a pharmaceutical formulation to an individual in order to alter a physiological state, whether or not the process includes a curative element.
(27) “Controlled release” of a drug or other bioactive material means release of the material in a pre-determined or adjustable way such that the amount or rate or timing of release is pre-set or is altered in a desired way.
(28) The terms “controlled release device”, “controlled release dosage form” and similar terms mean any formulation or device wherein the release rate (e.g., rate of timing of release) of a drug or other desired substance contained therein is controlled by the device or dosage form itself and/or by the environment of use. Controlled drug delivery includes delivery of an amount of drug to a particular target site at a particular time, for example delivery of a bolus of drug to a tumour site.
(29) “Sustained release” of a drug or other material means release over an extended period of time, for example minutes, hours or days, such that less than all the bioactive material is released initially. A sustained release rate may provide, for example, a release of a specified amount of a drug from a pharmaceutical formulation, over a certain time period, under physiological conditions or in an in vitro test.
(30) “Bolus” release means release of a large dose, for example substantially all of a drug at one time or over a short period of time. Bolus release can be preceded or followed by sustained release.
(31) The term “burst effect” is used, often in the context of drug delivery, to mean release of a bioactive material from a composition in an amount or at a rate which is higher than is desired (typically, in drug delivery, higher than the therapeutic window). A burst is generally followed by a rapidly decreasing rate of release. A burst effect may be defined as the release of more than a defined threshold proportion of the bioactive material over a defined time under defined conditions.
(32) The term “functionalized”, as applied to a chemical compound, including a polymer, means that the compound has been treated so that it contains a functional moiety (i.e. a moiety which will undergo a further desired chemical reaction) which was not present on the compound before the treatment, or so that the polarity of the compound is changed, as evidenced, for example, by a change in the solubility parameter.
(33) The term “alkyl” includes alkyl moieties which are straight chain alkyl moieties, branched chain alkyl moieties, cycloalkyl moieties, and moieties which consist essentially of two or more of straight chain alkyl, branched chain alkyl and cycloalkyl moieties.
(34) The term “bioerodable” (sometimes alternatively “biodegradable”) as applied to a QCYC carrier or to a release composition means that the carrier or composition, when placed in the human body, is eliminated from the human body, the carrier being eliminated without change or as one or more lower molecular weight products resulting from the degradation of the carrier in the human body. The elimination may take place relatively rapidly, e.g. in a period of up to 10 weeks, but is more often longer, for example over a period of up to 1 year or longer, e.g. up to 3 years. Carriers that are not bioerodable may leave the body by voiding if part of an oral formulation, or by explanation if part of an implanted formulation.
(35) Some of the structural formulas given below for QCYC polymers show the repeating units in the general form

-(-unit#1-)ₓ-(-unit#1-)_{y}-

This representation is used to denote polymers in which the different repeating units are distributed randomly and/or are distributed in blocks containing only one of the repeating units. Thus, the polymers represented by these formulas can be either random copolymers or block copolymers.

This specification incorporates by reference all documents referred to herein and all documents filed concurrently with this specification or filed previously in connection with this application, including but not limited to such documents which are open to public inspection with this specification.

The release material can be of any kind. Specific examples of release materials include:

(1) Drugs, defined as “a material which is biologically active in a human being or other mammal, locally and/or systemically, including any chemical moiety that can be used for diagnosis or in a diagnostic test”. The term “drug” includes food additives, including nutrients, used in food supplements for animal or human consumption, and antimicrobials (i.e. materials that attack bacteria and other microbes). Examples of drugs are disclosed in the Merck Index, the Physicians Desk Reference, in column 11, line 16, to column 12, line 58, of U.S. Pat. No. 6,297,337, and in paragraph 0045 of US 2003/0224974, the entire disclosures of which are incorporated by reference herein for all purposes

(2) Agricultural materials, defined as materials which, at the time of delivery or after delivery to an agricultural or aquacultural site, produce a desired effect at the agricultural or aquacultural site, for example on live vegetable matter (including seeds), or on live fish, or on the environment surrounding live vegetable matter or live fish, for example soil which surrounds a seed or in which a plant is growing. In many cases, the agricultural material is bioactive, for example is a biocide (e.g. a fungicide, herbicide, pesticide, mildewcide or insecticide). Agricultural materials can also be used in other contexts. For example mildewcides are widely used to control the growth of mildew and fungi in buildings and other sites. Specific examples of bioactive materials include Thiram, Fludioxaonil, Captan, Rival, and Apron and insecticides such as imidacloprid, chlothianidin, dinotefuran and thiomethoxam. More detailed disclosure of suitable agricultural materials is to be found in the PCT application filed Jun. 4, 2010, (Docket 011 PCT) claiming priority from U.S. application Ser. No. 12/455,650, filed Jun. 4, 2010 (Docket 011 US), incorporated by reference herein.

(3) Cosmetic materials, defined as materials which are not “drugs” as defined above and which, at the time of delivery or after delivery to the human body, change (generally improve or preserve) the appearance of, and/or hygiene of, and/or smell associated with, the human body (including the hair); cosmetic materials can be delivered in combination with materials which are “drugs” as defined above. Further details of cosmetic materials, and of cosmetic compositions, which are alternatively referred to as compositions for personal care , e.g. hair care, skin care, sun care, color care and body care, can be found for example in U.S. Pat. Nos. 6,540,984, 6,989,417, 7,101,832 and 7,175,832 (Landec Corporation); U.S. Pat. No. 5,736,125 (National Starch Corporation); U.S. Pat. Nos. 5,622,694, 5,662,892, 5,916,547, 5,919,439, 6,074,628 and 6,113,883 (Procter and Gamble Corporation); and U.S. Pat. Nos. 6,503,494, 6,572,869, 6,361,781, 6,569,409, 6,464,969, 6,565,839, 7,335,348, 6,789,550, 6,811,770, 6,949,504, 7,129,276, 7,255,870, 6,946,518, 7,090,420, 7,083,347, 2002041857, 2002127251, 2003039621, 2004005279, 2005188474, 2005191262, 2005172421, 2005191258, 2004180021, 2004191200, 2004228890, 2005031656, 2005013838, 2005142082, 2005123493, 2005008667, 2005031565, 2005169949, 2005169865, 2005261159, 2005175570, 2005180936, 2006078519, 2005287093, 2005287183, 2005287100, 2005287101, 2006078520, 2006130248, 2006233732, 2006216257, 2006292095, 2006263438, 2007134192, 2005137117 and 2003039671 (L'Oreal). The entire disclosure of each of those patents and patent publications is incorporated herein by reference for all purposes.

(4) Other materials, defined as materials which are not “drugs” or “agricultural materials” or “cosmetic materials” as defined above and which at the time of delivery provide a useful function to the environment into which or onto which the release material is provided, for example which is an organic or inorganic material which takes part in or catalyses a chemical reaction, a fragrance-emitting material for a vaporizer, or a lubricating material.

Additional disclosure of release materials can be found in the patents and applications incorporated by reference herein.

The compositions of the invention comprising a “supplementary CYC compound” or “mono-Cy CYC compound” and a release material can for example contain 0.1 to 5% or more, e.g. at least 5%, at least 8%, or at least 20%, by weight of the release material.

### Additional and Supplementary CYC Carriers

### 1. Crystalline Derivatives of Various Compounds.

### 1A. Crystalline Derivatives of Hyaluronic Acid.

Hyaluronic acid (HA) or hyaluronan is a naturally occurring polysaccharide, specifically, a glycosaminoglycan consisting of repeating disaccharide units (D-glucuronate and N-acetyl-D-glucosamine which can be modified so that it includes Cy moieties and thus becomes a QCYC carrier. HA contains hydroxyl groups and carboxyl groups for one or more of which can be partially or completely modified to produce a modified hyaluronic acid which contains one or more Cy moieties. The modification can be carried out in a single step, for example by alkylating one or more of the hydroxyl groups, e.g. by reacting HA with the anhydride or other suitable derivative of a Cy-containing acid.

In one example of such modification, HA, preferably a low viscosity HA, is partitioned in a non-reactive water miscible solvent and reacted with a Cy-containing anhydride, e.g. stearic anhydride in the presence of concentrated sulfuric acid to give a QCYC carrier.

In another example, HA is reacted with adipic dihydrazide, and the reaction product is modified to include Cy moieties, e.g stearyl or behenyl moieties.

In another example, HA is reacted with the reaction product of a diisocyanate and a Cy-containing alcohol or ethoxylated alcohol.

HA can also be modified so that it can be polymerized with one or more other monomers, oligomers or polymers, which optionally contain Cy moieties, thus producing a random, grafted or cross-linked polymer. For example, the hydroxyl groups of HA can be reacted with an anhydride or other suitable derivative of an unsaturated carboxylic acid or ester, and then copolymerized with a Cy-containing acrylate or methacrylate monomer.

In one example, methacrylic anhydride is mixed with powdered dry HA in the presence of a small amount of sulfuric acid to make a methacryoyl derivative of HA, and that derivative is then copolymerized with stearyl acrylate to provide a polystearyl acrylate polymer with a grafted hyaluronic acid moiety. In another example, HA is reacted with a Cy-containing amine, e.g. stearyl amine or behenyl amine—by reaction of the crystalline amine using a carbodiimide active ester mediated coupling reaction to provide a Cy moiety attached to the (5) carboxylic acid group on the D-glucuronate portion of the disaccharide repeating unit.

Injectable biodegradable hydrogels composed of HA-tyramine conjugates for use in drug delivery, dermal filling and tissue engineering have been disclosed by M. Kurisawa, joo Eun Chung, Yi Yan Yang, Shu Jun Gao, Hiroshi Uyama, in Chem Commun., 2005, 4312-4314. If a Cy-substituted tyramine is used together with unsubstituted tyramine in the preparation of the tyramine substituted HA or conjugated HA, a simple crystalline substituted, tyramine substituted HA is possible which can be injected as a low viscosity fluid and cross-linked inside the body (in-situ gelation) after injection to form a drug delivery depot, an injectible depot or used as an in-situ scaffolding device for tissue engineering and drug delivery for tissue engineering. This crosslinking of the HA-tyramine and crystalline substituted tyramine conjugate can for example be effected by injecting two solutions through syringes—the HA-tyramine and substituted tyramine solutions containing hydrogen peroxide in one syringe and horseradish peroxidase in the other syringe, the latter providing the catalyst to induce oxidative crosslinking or coupling of the unsubstituted tyramine moieties which had been earlier conjugated to the HA. Instead of HA, CMC-tyramine complex (CMC is carboxymethyl cellulose), a CMC-tyramine complex with both unsubstituted tyramine and crystalline alkylated tyramine as aforementioned is used to create an in-situ crosslinkable crystalline CMC to regulate through injectible depots delivery of active agents or application and in-situ gelation of a delivery of a crystalline CMC-tyramine complex holding active agents to the skin in a dermatological formulation.

A crystalline HA derivative can also be prepared by reacting a preformed acrylate ester of a Cy-containing polyol, for example, a stearyl monoglyceride, with the (5) carboxylic acid portion of the D-glucuronate ring of HA. Once the acrylate crystalline ester is formed, it can easily be crosslinked with a peroxide or by UV radiation to form an in-situ gel useful as a wound dressing, in tissue engineering or as an in-situ gel. The use of UV radiation is particularly attractive for application of this crystalline acrylate modified HA containing an active therapeutic agent to promote healing or provide an active drug at a wound site or for use in a tissue scaffolding application, thereby, providing a gelatinous hydrogel with active therapeutic for sustained release of the active material.

An advantage of using a hyaluronic acid derivative to deliver drugs is that HA is ubiquitous in the mammalian body. Consequently, such derivatives are useful for sustained release of a drug, for example, a small insoluble molecule or a protein.

It is well known that lower molecular weight fractions of HA, oligosaccharides of HA, seem to have therapeutic benefits in the treatment of cancer (Toole B. P., Wight T. N., Tammi M. (2002) J. Bio. Chem., 277, 4593-4596). Thus, the ability to prepare crystalline QCYC carriers containing low molecular weight fractions of HA, designated as crystalline HA oligosaccharides for use in transporting and targeting delivery of anti-cancer therapeutics to tumors either directly or in combination with unsubstituted hyaluronan oligosaccharides and other active therapeutics is possible. Multiple drugs of the oligosaccharides and other anti-cancer drugs, for example, methotrexate, paclitaxel, doxorubicin mixed with crystalline oligosaccharides may provide an attractive sustained release of these anti cancer drugs to a targeted tumour site.

### 1B. Crystalline Derivatives of Collagen and Gelatin.

Collagen and gelatin can also be converted to a crystalline QCYC carrier, for example by reacting a Cy-containing aldehyde, for example, stearaldehyde or behenaldehyde, with the collagen or gelatin to produce a crystalline collagen or gelatin for use in drug delivery, tissue engineering or dermatological formulating.

Like HA, collagen or gelatin may be modified by forming conjugated systems with tyramine and Cy-containing tyramine to provide different forms of crystalline collagen or gelatin for use in delivering active agents orally or by injection or used in a dermatological formulation. This preformed collagen or gelatin tyramine conjugate with both unsubstituted and crystalline Cy moiety substituted tyramine can be crosslinked by an in-situ peroxidase catalyzed oxidation of the unsubstituted tyramine conjugated on the collagen or gelatin.

### 1C. Crystalline Derivatives of Polysaccharides and Carbohydrates.

Like HA, polysaccharides and carbohydrates, e.g. pectin, alginate, alginic acid, chondroitin and chondroitin sulphate can be converted into crystalline derivatives.

### 1D. Crystalline Derivatives of Cellulosic Polymers.

Cy-containing cellulosic polymers can be prepared by esterifying cellulosic polymers containing hydroxyl groups, e.g. hydroxyethyl cellulose (HEC) and hydroxypropyl methyl cellulose (HPMC) with suitable Cy-containing compounds. Other ways of attaching Cy moieties to cellulosic polymers can be used. For example, a cellulosic polymer containing hydroxyl groups can be reacted with the reaction product of a Cy-containing alcohol with a diisocyanate, the reaction product having remaining free isocyanate groups.

### 1E. Crystalline Derivatives of Cyclodextran.

A Cy-containing cyclodextran can be prepared by alkylation of a beta dextran to create either a QCYC carrier polymer or a mono-Cy CYC compound which by its crystalline nature may self assemble with other CYC carriers or other mono-Cy CYC compounds (stearyl alcohol as an example) for use in delivering active agents in, for example, drug delivery or tissue engineering applications.

### 2. QCYC Carriers Containing Metal Ion Complexes (MOFs)

A QCYC carrier which contains carboxylic or other acid groups can be modified by reaction with a source of a multivalent (e.g. divalent or trivalent) cation, e.g. Ca++ or Mg++, to form a gel-like or lattice-like structure. This structure is referred to herein as a “metal organic framework” or “MOF”. Specific examples of QCYC carriers suitable for such modification include SSP polymers, ECC polymers, particularly ECC PLGA polymers, modified hyaluronic acid, and other natural polymers which have been modified to contain Cy moieties. Release materials can be captured within the MOF and will be released in a controlled way, and more slowly than they would be if the MOF was not formed. For example, a therapeutic quantity of the drug may be delivered over a period of at least 30 days, or for a lesser time, for example 10-14 or 4-7 days, or 4-24 hours. In some cases, this reduces the time between injections (which is generally inconvenient and/or painful) of drugs which must be delivered by injection because the GI tract cannot absorb them, for example acid-sensitive protein or peptide drugs.

Calcium phosphate and calcium carbonate are examples of suitable sources of cations. When the MOF is to be used to deliver a drug, the cation must be one which is compatible with the mammal to which the drug is to be delivered.

Examples of drugs which can be delivered from MOFs (and also from other QCYC carriers) include peptides, proteins, siRNA molecules, monoclonal, synthetic or natural antibodies, e.g. with a molecular weight of at least 150,000 Kdal, foldamers or peptoids (which are beta amino acids which can act as antimicrobial and/or anti viral agents), small molecules such as highly toxic cisplatin, a chimeric protein comprising, for example, the Fc portion of an immunoglobulin and the functional (binding) domain of another molecule (for example, an anti-inflammatory cytokine or other immunoregulatory TNF or a TNF binding molecule such as a TNF receptor), a molecule that binds to TNF or an antagonist of TNF which binds competitively with TNF at TNF binding sites, an anti-inflammatory molecule; and Etanercept, Infliximab, Enbrel, Remicade and variants, derivatives, analogues and functional equivalents thereof.

### 3. Mono-Cy CYC Carriers

Mono-Cy CYC carriers are defined above. The molecular weight of the mono-Cy CYC carriers is typically 200-1000, preferably 250-900. Particularly having regard to the use of polymers as the delivery vehicles for bioactive materials, it is surprising that association of a release material with a mono-Cy carrier can be effective in controlling the rate at which the release material is released.

Mono-Cy CYC carriers include for example Cy-containing carboxylic acids; monoglycerides, diglycerides and triglycerides of Cy-containing carboxylic acids; Cy-containing alcohols; monoesters of hydroxyl-containing compounds, for example, monoesters of polyols, e.g. glycerin, in which one of the hydroxyl groups has been esterified by a Cy-containing moiety, for example, a crystalline monoester of 1,3-propanediol, a crystalline monoester of pentaerythritol, a crystalline ester or crystalline alkylated ether of an alpha glucoside, a crystalline monoester of a soluble carbohydrate alcohol, a crystalline monoester of a hydroxy-containing carboxylic acid, e.g. lactic acid, glycolic acid, succinic acid, hydroxypropionic acid, tartaric acid, citric acid, and malic acid. The corresponding polyesters of polyols are examples of the CYC compounds disclosed in the earlier applications.

The mono-Cy CYC carriers can also contain polyoxyalkylene, e.g. polyoxyethylene or polyethylene glycol moieties, e.g. an ethoxylated crystalline ester, an ethoxylated crystalline fatty alcohol, an ethoxylated mono- or diglyceride, an ethoxylated polycarboxylic acid, for example ethoxylated tartaric acid. Mono-Cy CYC carriers containing ethoxylated crystalline Cy moieties are also useful up for self assembly into CYC assemblies with HA derivatives that are either crystalline or pegylated (HA-PEG). For example, HA may be ethoxylated to give a pegylated HA molecule containing polyoxyethylene chains on a few or many of the repeating disaccharide linkages of the HA. These substituted HA polymers can associate with Cy-containing alcohols or ethoxylated alcohols by interaction of the hydrophilic polyethylene glycol chains of the PEG substituted HA with the crystalline fatty alcohol ethoxylate alcohol of the pegylated HA. This form of association will lead to sustained release of an active therapeutic mixed into the polymeric formulation. The rate of release will depend upon the amount of association of the Cy-containing alcohol ethoxylate with the pegylated HA. Also, the presence of the mono-Cy CYC carrier enables easier processing of the HA derivatives with the active ingredient, active therapeutic or drug molecule at lower viscosity mixing, lower temperature processing or higher drug loading capability.

Mono-Cy CYC carriers are useful for example, for delivering an acid-sensitive drug like a peptide or protein or a drug that can damage the stomach like aspirin or ibuprofen, because their low molecular weight enables them to release the drug quickly once in the gastrointestinal tract, particularly if the mono-Cy CYC carrier is a crystalline acid material such as a fatty acid like stearic acid, or a monoester of citric acid. They are also useful for delivering drugs that are highly acidic or deleterious to the stomach, because they provide an effective way to transport stomach-sensitive drugs through the stomach for absorption in the gastrointestinal tract. The crystallinity of the mono-Cy carrier shields the acid sensitive therapeutic during the time the active ingredient is in the low pH stomach.

In preferred cases, mono-Cy CYC carriers can enable sustained release of a drug, with little or no initial burst. In some cases, it is possible to provide close to zero order release characteristics for time periods of several hours, a day, several days and up to 2 weeks and longer, and even up to 1-2 months.

Another attribute of many mono-Cy CYC carriers is that they can be easily mixed with release materials. For example, the mono-Cy CYC carrier and the drug or other release material can be mixed without a solvent at a temperature above the Tp of the mono-Cy CYC carriers, enabling higher loading of active therapeutic, at a lower processing viscosity and a lower processing temperature, highly attractive for mixing temperature sensitive drug molecules like biologics—peptides and proteins whose conformational structure is often affected by processing temperatures.

Compositions comprising mono-Cy CYC carriers and release materials can contain other ingredients, for example, relatively small amounts, generally less than 20%, preferably less than 10%, for example 2-20% or 4-10%, of modifying polymers, which will adjust the release properties. Examples of such modifying polymers include polymeric QCYC carriers, including ECC polymers, bioerodible polyesters, e.g. polytrimethylene carbonate, PLGA, PLA, PGA and polycaprolactone, PVP, polyvinyl alcohol, polyvinyl acetate, polyacrylates, polyacrylic acid, polyethylene glycol. The compositions can include more than one mono-Cy CYC carriers, in order to achieve release of the same or different release materials in a desired way. The compositions can also contain a small amount, typically 10% or less, of a hydrogel polymer like an N-isopropyl acrylamide polymer or a polyhydroxyethyl acrylate polymer. If a hydrogel contains Cy moieties, the presence of a mono-Cy CYC carrier will reduce the tendency of hydrogels to exhibit a burst effect.

Any of the above concepts can be employed to make “self-assemblies” which contain crystalline moieties (“CYC assemblies”) as taught by prior reference to the earlier patent applications. These self assemblies provides ways to vary the release properties of the therapeutic ingredient in the self assembly mixture.

### Compositions Comprising Release Materials Containing Covalently Linked Cy Moieties

Compositions having desirable release properties can be prepared by mixing a release material which contains at least one covalently linked Cy moiety with a QCYC carrier, e.g. a mono-Cy CYC carrier. The Cy moiety can be an integral part of a release material or it can be introduced by modification of a release material, thus creating for example a pro-drug or conjugated drug. The Cy moiety can for example be attached covalently to the drug or other release material through an ester, carbonate, carbamate or anhydride linkage or other linkage, which, if desired, will be ruptured in the environment at the delivery site (e.g. the body, the mouth or the stomach). The Cy moiety of the release material will self assemble with the QCYC carrier, resulting in release properties which are different from those obtained when the release material is delivered on its own and different from those obtained when the release material is delivered as a composition with a carrier which is not a QCYC carrier. Also, the Cy moiety covalently attached to a active ingredient can be mixed with a mono-Cy CYC carrier to provide an easy to process formulation of an active ingredient with a crystalline Cy active ingredient.

In general, the use of a pro-drug or other modified release material with a QCYC carrier is advantageous when slow release of the release material is desired. However, in some cases, an ester bond may be hydrolyzed in a short time as a result of the environment into which it is placed. For example, a crystalline ester of ibuprofen in the GI tract may hydrolyze adequately within a 24 hour period, a typical residence time for materials passing through the upper GI tract. Or, in the case of an anhydride linkage between the drug and QCYC carrier, the release rate will be influenced by the time required for moisture to penetrate the crystalline QCYC non-polymer linkage to allow hydrolysis of the anhydride linkage. This type of anhydride pro-drug with the crystalline QCYC non-polymer attached enables another method of control of the release once hydrolyzed as the crystalline QCYC non-polymer will help regulate release of the hydrolyzed active therapeutic from the mixture of the active therapeutic and the crystalline QCYC non-polymer. This mode of delivery would be very attractive for oral administration of an active therapeutic either in the mouth, stomach or the GI tract.

Other examples of modified release materials include Cy-containing derivatives of doxorubicin, methotrexate, paclitaxel and docetaxel, for example a monoester of stearic acid with doxorubicin or a monostearyl ester of methotrexate. As aforementioned the release properties of these Cy-containing prodrugs may be modified by mixing with mono-Cy CYC carriers. These formulations can be administered orally, as a microparticle in an injectible dosage form, or as a component of a dermatological or cosmetic skin formulation.

As significant benefit is the preparation of prodrug or conjugated drugs of a Cy moiety with, for example, an anti-cancer drug like doxorubicin or paclitaxol covalently attached to a QCYC carrier of crystalline HA, the crystalline HA controlling the release of the conjugated active by controlling the rate of hydrolysis of the drug away from the crystalline Cy moiety. The conjugation of typically toxic anti-cancer drugs is particularly attractive for targeted delivery of these active therapeutics to a targeted tumour site. HA selectively recognizes a transmembrane receptor—CD44—over-expressed in most primary cancers and associated with tumour growth and progression. Thus the combination of a targeting material with a Cy moiety to prevent release of the active therapeutic until in the vicinity of the tumour site.

It must also be recognized that HA and the anti-cancer drug may be mixed in a matrix form and release controlled by the addition of a QQCYC carrier, a mono-Cy CYC carrier or a mixture of QCYC carriers into self assemblies to control the delivery of the anti-cancer therapeutic to the targeted site. The delivery of an active anti-cancer therapeutic in a HA containing crystalline matrix is highly attractive as a targeting device because of the CD44 expressed receptor associated with cancer cells.

A crystalline prodrug may also be prepared reacting any of the aforementioned crystalline long chain alcohols or alcohol ethoxylated alcohols with a diisocyanate and subsequently reacting the crystalline isocyanate with a drug having a free hydroxy group.

Another example is a peptoid (some of which are strong antimicrobial antibiotics) which has been modified covalently or ionically (see the next section) and mixed with a QCYC carrier for use as a long-lasting antimicrobial for wall coatings additives, floor wax additives, door knob coating additives, hospital hardware and furniture coating additives.

Other long-lasting antimicrobial compositions can likewise be prepared by modifying the antimicrobial covalently or ionically.

Examples of modified agricultural release materials are (1) a compound obtained by transesterification of the fungicide metalaxyl with Cy-containing alcohol, e.g. stearyl alcohol; (2) zinc dimethyl dithiocarbamate (the fungicide Vancide MZ-96), modified by a Cy-containing compound; (3) Glyphosate, N-(phosphonomethyl) glycine (Round-Up), esterified with a Cy-containing alcohol, e.g. stearyl alcohol.

Cysteine, an amino acid food supplement for animal nutrition, whether modified or not modified can be mixed with a QCYC carrier to regulate its uptake in the animal digestive tract. Similarly, mixing an antibiotic used in feed additives with a QCYC carrier may reduce the quantity of antibiotic required.

In addition, the preparation of a Cy-containing pro-drug can be advantageous in order to isolate an active therapeutic in for use in a treatment. For example, a siRNA generated in picomole quantities can be reacted via succinoylation or through disulfide linking between a QCYC carrier and the siRNA, thereby providing a slow-to-release siRNA material which also has the advantage of being stable in the human environment, slowly releasing siRNA as body fluids break the covalent bond or interfere with the ionic association between the siRNA and the QCYC carrier.

### Compositions Comprising Release Materials Containing Ionically Linked Cy Moieties

Compositions having similarly desirable release properties can also be prepared by mixing a release material which contains at least one ionically linked Cy moiety with a QCYC carrier, e.g. a mono-Cy CYC carrier. The Cy moiety can be an integral part of a release material or it can be introduced by modification of a release material, thus creating for example a pro-drug. For example, a Cy-containing amine may be associated ionically with a drug containing an organic carboxylic group, thus forming a crystalline quaternary ammonium salt; or a Cy-containing carboxylic acid chloride or similar derivative may quaternize an amine-containing drug or other release material. For example, cisplatin or an analog thereof, can be modified by replacing the ammine groups in cisplatin by a Cy-containing amine, thus providing a slow release cisplatin. The modified cisplatin may be for example distearyl amine dichloroplatin. In another example, acetate groups in satraplatin may be replaced by a Cy-containing carboxylic acid, producing for example, distearoyl dichloro cyclohexylamine Pt(IV) ammine.

In the agricultural field, Imidacloprid (a broad spectrum insecticide for crop protection) can be modified by mixing it with a Cy-containing compound which will associate with it ionically, for example, stearic acid, a stearyl amine or stearyl quaternium amine, a stearyl alcohol or a lightly sulphated but still crystalline tallow to provide a combination fast release with additional slow release imidacloprid. The mixture also would have the benefit of less dusting during handling. In another example, glyphosate, N-(phosphonomethyl) glycine (Round-Up) is modified with a crystalline fatty amine to control the release of the glyphosate in the ionically associated crystalline matrix. By varying the formulation content of the QCYC polymer carrier and the mono-Cy CYC compound, formulations with different sustained release of active ingredients are possible.

Also, crystalline fatty amines can form salts with the (5) carboxylic acid of D-glucuronate molecule of the disaccharide link of HA to provide an ionically attached crystalline moiety to the HA creating a QCYC carrier for controlling the release of an active ingredient. Other acid containing polysaccharides, for example, pectin, alginate, chondroitin, chondroitin sulfate may be used to form the QCYC carrier with the crystalline Cy moiety, for example, stearyl or behenyl amine. Also, collagen or gelatin maybe used to form ionic associative materials with the crystalline fatty amines. In addition, CMC (carboxymethyl cellulose) may be used to form the crystalline ionic complex for formulation with an active therapeutic and, optionally, a mono-Cy CYC compound to control the sustained release of an active therapeutic.

Also, crystalline beef tallow can be physically mixed with urea and other fertilizers and/or nutrients to create a slow release fertilizer product.

In the cosmetic field, an ionic association of a Cy-containing quaternary ammonium with a cosmeceutical or neutraceutical release material will help to regulate the release of the release material. In another example, a peroxide which has optionally been modified to contain a Cy moiety and which is mixed with a QCYC carrier will release the peroxide at a slower rate, for example in the treatment of acne, and could be used in place of the fast-acting benzoyl peroxide, salicylic acid or other active ingredient in many existing formulas. In an antiseptic hand wash, the rate at which an emollient is released can be reduced by mixing the emollient, which optionally contains or has been modified to contain a Cy moiety, with a QCYC carrier. In this manner, the Cy-containing residue which remains often handwashing will provide emollient properties and retain moisture in the skin.

In another example, a cosmetic material, e.g. a sunscreen, antioxidant sun care materials or skin care material, can be modified by covalently attaching a Cy moiety to it. Another example is a cosmetic material, e.g. an oil, which has been modified by attaching a Cy-containing moiety to it through an anhydride linkage. Such a modified material can be part of an anhydrous composition for controlled release of the oil or other material to the skin as the anhydride linkage is slowly hydrolyzed by moisture. In another example, a long-lasting odor or fragrance can be produced by mixing it with a QCYC carrier, optionally after covalently or ionically modifying the odor or fragrance so that it contains a Cy moiety. In another example, Botox or a modified Botox, is mixed with a QCYC carrier, preferably a biodegradable polymer, e.g., an ECC-PLGA.

### Use, Without a QCYC Carrier, of Release Materials Containing Cy Moieties

Release materials which have been modified so that they contain Cy moieties, as described in the foregoing sections, can be used in compositions which do not contain a QCYC carrier, and when so used often have different (and for most purposes better) release properties than the release materials before modification. Release materials which are used without a QCYC carrier have a crystalline melting temperature, Tp, of least 0° C., e.g. at least 25° C., e.g. 27-100° C., and a ΔH of at least 3 J/g. The invention includes, therefore, such release materials, and the use of such release materials even when they are not associated with a QCYC carrier.

### Use of Combinations of Modified and Unmodified Release Materials

Unmodified release materials and modified release materials, as described in the foregoing sections, can be used in combination, with one or other or both or neither being associated with a QCYC carrier, to achieve release of the active material over a desired schedule. For example, the stearyl ester of glyphosphate, the ionically associated glyphosate and unmodified glyphosate can be mixed together to provide a long-lasting weed killer product.

### Further Uses of QCYC Carriers

Any of the concepts discussed above for specific release materials can be employed for other release materials, including for example fragrances, odors and releasing reactants.

### Use of Biodegradable, QCYC Carriers for Liquid Absorption

The biodegradable QCYC carriers, in particular, the ECC PLGA polymers, are useful for the absorption of liquids, particularly aqueous liquids, whether or not a release material is associated with the QCYC carrier. They can be used, for example, to replace or supplement the highly absorbent polyacrylates, polyacrylamides and cross-linked polyacrylic acids, which are not biodegradable, and which are presently used in a wide range of products, in particular diapers. The proportion of Cy-containing moieties in the QCYC carrier can be adjusted to storage and use requirements, followed by biodegradation under expected conditions of, for example, pH, time, temperature and extent of hydration.

1. A composition which comprises
(1) a mono-Cy CYC carrier, and
(2) a release material,
the mono-Cy CYC carrier being a non-polymeric compound which
(A) has crystallinity such that the mono-Cy CYC carrier has a crystalline melting temperature, Tp, of least 0° C. and a ΔH of at least 3 J/g, measured as hereinbefore defined, and
(B) has the formula.
Q-Cy
wherein
Cy is a moiety which provides the mono-Cy CYC carrier with its crystallinity, and
Q is a moiety which (i) is free of Cy moieties and (ii) contains at least one functional moiety which associates with the release material.

2. A composition according to claim 1 wherein the release material contains a Cy moiety.

3. A composition according to claim 1 wherein the functional moiety is a hydrophilic moiety and the release material contains a hydrophilic moiety.

4. A composition according to claim 1 wherein the functional moiety comprises a polyoxyalkylene group.

5. A composition according to claim 1 wherein the functional group forms a covalent link with the release material.

6. A composition according to claim 1 wherein the functional group is a polar group.

7. A composition according to claim 1 wherein the mono-Cy CYC carrier has a Tp of 27-100° C.

8. A composition according to claim 1 which comprises microparticles having an average particle size of 0.5-250μ.

9. A composition according to claim 1 which contains 2-10% of a CYC polymer as hereinbefore defined

10. A method of delivering a release material which comprises exposing a composition as claimed in claim 1 to an aqueous environment at a temperature below Tp.

11-13. (canceled)

14. A composition which comprises.
(1) a reaction product obtained by reacting a compound containing a Cy moiety with a compound selected from the group consisting of hyaluronic is acid, collagen, gelatin, a polysaccharide, a carbohydrate, and cyclodextran, thereby producing a reaction product containing a covalently linked Cy moiety, and
(2) a release material.

15. A composition according to claim 14 wherein the release material contains a Cy moiety.
